(19) United States
(12) Patent Application Publication
(10) Pub. No.: US 2021/0032692 A1
(43) Pub. Date: Feb. 4, 2021

(54) METHODS OF DETERMINING DONOR CELL-FREE DNA WITHOUT DONOR GENOTYPE

(71) Applicant: TAI Diagnostics, Inc., Wauwatosa, WI (US)

(72) Inventors: Aoy Tomita Mitchell, Elm Grove, WI (US); Karl Stamm, Brookfield, WI (US)

(73) Assignee: TAI Diagnostics, Inc., Wauwatosa, WI (US)

(21) Appl. No.: 16/639,489

(22) PCT Filed: Aug. 17, 2018
(86) PCT No.: PCT/US18/00278

§ 371 Date: Feb. 14, 2020

Related U.S. Application Data
(60) Provisional application No. 62/547,098, filed on Aug. 17, 2017.

(51) Int. Cl. C12Q 1/6858; C12Q 1/6851; C12Q 1/6883

(52) U.S. Cl. CPC C12Q 1/6858; C12Q 1/6851; C12Q 2531/113; C12Q 2537/165; C12Q 2535/125; C12Q 1/6883

(57) ABSTRACT

This invention relates to methods and systems for assessing an amount of non-subject nucleic acids, such as donor-specific cell-free DNA, in a sample from a subject. The methods and systems can include the simulation of non-subject genotype when unknown. The methods and systems provided herein can be used to determine risk of a condition, such as transplant rejection.

### RELATED APPLICATIONS

This application claims the benefit under 35 U.S.C. § 119(e) of the filing date of U.S. Provisional Application 62/547,098, filed Aug. 17, 2017, the contents of which is incorporated by reference herein in its entirety.

### FIELD OF THE INVENTION

This invention relates to methods and systems for assessing an amount of non-subject nucleic acids, such as donor-specific cell-free DNA, in a sample from a subject. The invention provides systems for analyzing and/or assessing an amount of non-subject nucleic acids in a sample from a subject without non-subject genotype information. The methods, compositions, and systems provided herein can be used to determine risk of a condition, such as transplant rejection.

### SUMMARY

The present disclosure is based, at least in part, on the surprising discovery of methods of determining amounts of cell-free DNA, such as non-subject and/or subject cell-free DNA, without the need for knowledge of the non-subject genotype. Described are these methods and systems for the quantification of cf-DNA in subjects, such as transplant subjects, that can be used as a noninvasive assay, such as for the diagnosis of acute rejection and/or clinically significant adverse events, without the need to know the non-subject genotype (e.g., donor genotype). The methods and systems can also be used to determine subjects at low or high risk, such as of rejection and/or clinically adverse events. The methods and systems can also be used to monitor any of the subjects provided herein. In some embodiments, the methods and systems employ a simulation (e.g., Monte Carlo simulation) of non-subject genotype (e.g., donor genotype). Such methods and systems can be employed in any instances where the sample is of mixed genotypes and the non-subject genotype is not known. The examples and text that refer to the scenario of transplant subjects is for exemplification, and is not intended to imply that the assays must be so limited.

In one aspect, a method of determining an amount of non-subject nucleic acids in a sample from a subject is provided. In some embodiments, the method comprises analyzing amounts of alleles at multiple respective targets in a sample, and identifying quantifiable and/or informative targets, within the sample, performing simulations with possible genotypes for a non-subject; and determining amounts of alleles of each target attributed to the non-subject and, optionally, the subject, based on possible or probable non-subject genotype(s) determined from the simulation, and, optionally, determining an amount (e.g., percent or ratio) of non-subject to subject cf-DNA in the sample.

In one embodiment of any one of the methods or systems provided herein, the method or system further comprises determining the subject genotype. In one embodiment of any one of the methods or systems, the method or system further comprises performing amplifications to determine the amounts of alleles. In one embodiment of any one of the methods or systems, the method or system further comprises performing sequencing assays to determine the amounts of alleles.

In one embodiment of any one of the methods or systems, the sequencing assays or amplifications are performed for at least 30, 40, 50, 60, 70, 80, 90, or more targets.

In one embodiment of any one of the methods or systems provided herein, the method or system further comprises calculating quality measures on determined amounts (e.g., percents or ratios) in the sample. The quality measure of any one of the methods or systems can be any one of the quality measures provided herein or otherwise known in the art.

In one embodiment of any one of the methods or systems provided herein, the method or system comprises simulating a likely or possible non-subject genotype space.

In one embodiment of any one of the methods or systems provided herein, simulations (e.g., Monte Carlo simulations) are performed to determine a range of possible or probable genotypes for the non-subject.

In one embodiment of any one of the methods or systems provided herein, the method or system further comprises adjusting measured contributions for respective targets based on respective possible or probable genotypes (e.g., doubling measured contribution value responsive to determining the non-subject probable genotype is heterozygous).

In one embodiment of any one of the methods or systems provided herein, the method or system further comprises calculating an average, such as a mean or median, amount, such as a percent or ratio.

In one embodiment of any one of the methods or systems provided herein, the method or system further comprises determining each standard curve and/or sample amplification value meets a confidence threshold. In one embodiment of any one of the methods or systems provided herein, the method or system further comprises determining confidence values based on analysis of at least one of a historic amplification shape, specificity of the allele-specific PCR assay (e.g., with respect to a second allele), signal to noise ratio for a sample, slope and r-square value for standard curve sets, non-amplification values obtained on inserted controls, or contamination values obtained on the sample from negative controls.

In one embodiment of any one of the methods or systems provided herein, the method or system further comprises fitting data obtained from the sample to a historic amplification shape.

In one embodiment of any one of the methods or systems provided herein, the method or system further comprises determining the slope and r-square value for the standard curve sets does not exceed a threshold value.

In one embodiment of any one of the methods or systems provided herein, the method or system further comprises establishing a label for the non-subject or subject at each target identified as quantifiable and/or informative in the sample. In one embodiment of any one of the methods or systems provided herein, the method or system further comprises determining quantifiable and/or informative targets within the sample responsive to classifying a respective target according to genotype. In one embodiment of any one of the methods or systems provided herein, the method or system further comprises classifying the respective target as quantifiable and/or informative responsive to determining the subject and non-subject have different genotypes (e.g., the subject is homozygous for one allele and the non-subject is not homozygous or homozygous for the other allele). In one embodiment of any one of the methods or systems provided herein, the method or system further comprises adjusting measured contributions for a respective target responsive to determining the non-subject is heterozygous (e.g., doubling measured contribution value responsive to determining the non-subject is heterozygous).

In one embodiment of any one of the methods or systems provided herein, the method or system further comprises calculating a mean or median of informative (e.g., identified by the genotyping component) and quality-control-passed (e.g., identified by the quality control component) allele amounts (e.g., percent or ratios) and stores the mean or median values as the amount (e.g., ratio or percentage). In one embodiment of any one of the methods or systems provided herein, the method or system further comprises calculating a regularized robust coefficient of variation (“rCV”) based on a distribution of the informative and/or quantifiable targets and associated amounts (e.g., percents or ratios). In one embodiment of any one of the methods or systems provided herein, the method or system further comprises calculating a robust standard deviation (“rSD”) based on a median absolute divergence from a median minor species proportion. In one embodiment of any one of the methods or systems provided herein, the method or system further comprises converting the rSD into rCV by division with, for example, the non-subject cf-DNA amount (e.g., ratio or percentage). In one embodiment of any one of the methods or systems provided herein, the method or system further comprises adjusting rSD to avoid division by zero (e.g., by adding a quarter of one percent to the divisor). In one embodiment of any one of the methods or systems provided herein, the method or system further comprises identifying a sample suitable for quantification based on a threshold rCV value determined on a distribution of the informative and/or quantifiable targets and associated amounts (e.g., percents or ratios).

In one embodiment of any one of the methods or systems provided herein, the or system further comprises evaluating an average minor allele proportion of subject homozygous and non-quantifiable and/or non-informative targets against a contamination threshold.

In one embodiment of any one of the methods or systems provided herein, the method or system further comprises calculating a discordance quality check (“dQC”) value based on the average minor allele proportion of the subject homozygous and the non-quantifiable and/or non-informative targets and evaluate the dQC value against a threshold. In one embodiment of any one of the methods or systems provided herein, the method or system further comprises identifying samples suitable for quantification based on identifying a dQC value below a threshold, e.g., 0.5%.

In one embodiment of any one of the methods or systems provided herein, the non-subject is a donor. In one embodiment of any one of the methods or systems provided herein, the sample is from a transplant subject. In one embodiment of any one of the methods or systems provided herein, the transplant subject is a heart transplant subject. In one embodiment of any one of the methods or systems provided herein, the sample is from a pediatric subject. In one embodiment of any one of the methods or systems provided herein, the sample is from a pregnant subject.

In one embodiment of any one of the methods or systems provided herein, the method or system further comprises selecting an aggregate and/or the 95% confidence interval of the possible or probable simulations. In one embodiment of any one of the methods or systems provided herein, the method further comprises selecting simulations with below median dQC and rCV and/or determining the 95% confidence interval.

Provided herein, in another aspect, is a system for analyzing a sample from a subject, wherein the system comprises at least one processor operatively connected to a memory; a first component (e.g., a quality control component), executed by the at least one processor, configured to analyze (e.g., quantitative genotyping (“qGT”)) amounts of alleles at multiple respective targets in a sample, and identify quantifiable and/or informative targets, within the sample; a second component (e.g., a modelling component) configured to simulate possible genotype information for a non-subject; and a third component (e.g., a genotyping component), executed by the at least one processor, configured to determine amounts of alleles of each target attributed to the non-subject and, optionally the subject, based on possible or probable non-subject genotype(s) determined from the simulation, and, optionally, determining an amount (e.g., percent or ratio) of non-subject to subject amounts in the sample.

In one embodiment of any one of the systems provided herein, the system further comprises a fourth component (e.g., an analytic component), executed by the at least one processor, configured to calculate quality measures on determined amounts (e.g., percents or ratios) in the sample.

In one embodiment of any one of the systems provided herein, the third component is configured to simulate a likely or possible non-subject genotype space. In one embodiment of any one of the systems provided herein, the third component is configured to execute a simulation (e.g., Monte Carlo simulation) to determine a range of possible or probable genotypes for the non-subject. In one embodiment of any one of the systems provided herein, the third component is configured to adjust measured contributions for respective targets based on respective possible or probable genotypes (e.g., doubling measured contribution value responsive to determining the non-subject possible probable genotype is heterozygous).

In one embodiment of any one of the systems provided herein, the at least one processor is configured to calculate an average, such as a mean median, amount (e.g., percent or ratio).

In one embodiment of any one of the systems provided herein, the first component is configured to determine each standard curve and/or sample amplification value meets a confidence threshold. In one embodiment of any one of the systems provided herein, the first component is configured to determine confidence values based on analysis of at least one of a historic amplification shape, specificity of the allele-specific PCR assay (e.g., with respect to a second allele), signal to noise ratio for a sample, slope and r-square value for standard curve sets, non-amplification values obtained on inserted controls, or contamination values obtained on the sample from negative controls. In one embodiment of any one of the systems provided, the first component is configured to fit data obtained from the sample to a historic amplification shape. In one embodiment of any one of the systems provided, the first component is configured to determine the slope and r-square value for the standard curve sets does not exceed a threshold value.

In one embodiment of any one of the systems provided herein, the first or third component is configured to establish a label for the non-subject or subject at each target identified as quantifiable and/or informative in the sample. In one embodiment of any one of the systems provided, the first or third component is configured to determine quantifiable and/or informative targets within the sample responsive to classifying a respective target according to genotype. In one embodiment of any one of the systems provided, the third component is configured to classify the respective target as quantifiable and/or informative responsive to determining the subject and non-subject have different genotypes (e.g., the subject is homozygous for one allele and the non-subject is not homozygous or homozygous for the other allele).

In one embodiment of any one of the systems provided herein, the third component is configured to adjust measured contributions for a respective target responsive to determining the non-subject is heterozygous (e.g., doubling measured contribution value responsive to determining the non-subject is heterozygous). In one embodiment of any one of the systems provided herein, the third component calculates a mean or median of informative (e.g., identified by the genotyping component) and quality-control-passed (e.g., identified by the quality control component) allele ratios and stores the median values as an amount (e.g., the ratio or percentage).

In one embodiment of any one of the systems provided herein, any one of the components (e.g., the analytic component) is configured to calculate a regularized robust coefficient of variation (“rCV”) based on a distribution of the informative and/or quantifiable targets and associated amounts (e.g., percents or ratios). In one embodiment of any one of the systems provided herein, any one of the components (e.g., the analytic component) is configured to calculate a robust standard deviation (“rSD”) based on a median absolute divergence from a median minor species proportion. In one embodiment of any one of the systems provided herein, any one of the components (e.g., the analytic component) is configured to convert the rSD into rCV by division with, for example, the non-subject cf-DNA amount (e.g., percentage or ratio). In one embodiment of any one of the systems provided, the component is configured to adjust rSD to avoid division by zero (e.g. by adding a quarter of one percent). In one embodiment of any one of the systems provided herein, the system is configured to identify a sample suitable for quantification based on a threshold rCV value determined on a distribution of the informative and/or quantifiable targets and associated amounts (e.g., percents or ratios). In one embodiment of any one of the systems provided herein, the system is configured to evaluate an average minor allele proportion of subject homozygous and non-informative targets against a contamination threshold.

In one embodiment of any one of the systems provided herein, the system is configured to calculate a discordance quality check (“dQC”) value based on the average minor allele proportion of the subject homozygous and the non-quantifiable and/or non-informative targets and evaluate the dQC value against the threshold. In one embodiment of any one of the systems provided, the system is configured to identify samples suitable for quantification based on identifying a dQC value threshold, e.g., below 0.5%.

In one embodiment of any one of the systems provided herein, the system is further configured to select an aggregate and/or the 95% confidence interval of the possible or probable simulations.

In one embodiment of any one of the systems provided herein, the system is further configured to select simulations with below median dQC and rCV and/or determining the 95% confidence interval.

In one aspect, a report comprising any one or more values that result from any one of the methods or systems described herein is provided.

Provided herein, in another aspect, is a method of treating a subject. The method comprises evaluating a subject based on any one or more values that result from any one of the preceding methods or systems, and treating, recommending a treatment, changing a treatment, further monitoring or recommending further monitoring of the subject.

In one embodiment, any one of the embodiments for the methods provided herein can be an embodiment for any one of the compositions, systems, or reports provided herein. In one embodiment, any one of the embodiments for the systems provided herein can be an embodiment for any one of the compositions, methods, or reports provided herein.

### BRIEF DESCRIPTION OF FIGURES

The accompanying figures are not intended to be drawn to scale. The figures are illustrative only and are not required for enablement of the disclosure.

FIG. 1A shows the experimental determination of a threshold point (“cutpoint”) for CR2 with donor genotype information.

FIG. 1B shows the experimental determination of a threshold point (“cutpoint”) for CR2 without donor genotype information.

FIG. 2A shows the experimental determination of a threshold point (“cutpoint”) for graft vasculopathy with donor genotype information.

FIG. 2B shows the experimental determination of a threshold point (“cutpoint”) for graft vasculopathy without donor genotype information.

FIG. 3 is a block diagram of an example embodiment of a sample analysis system.

FIG. 4 is a block diagram of an example distributed computer system on which various aspects and functions of the disclosure are practiced.

FIG. 5 is a block diagram of a sample analysis platform, according to one embodiment.

### DETAILED DESCRIPTION

Accordingly, various aspects provide techniques to detect, analyze, and/or quantify nucleic acids (e.g., cell-free DNA), such as non-subject nucleic acids (e.g., non-subject cell-free DNA), in samples obtained from a subject. As used herein, “non-subject nucleic acids” refers to nucleic acids that are from another source or are mutated versions of a nucleic acid found in a subject (with respect to a specific sequence, such as a wild-type sequence). “Subject nucleic acids” therefore, are nucleic acids that are not from another source and are not mutated versions of a nucleic acid found in a subject (with respect to a specific sequence, such as a wild-type sequence). As used herein, any one of the methods or systems provided herein can be used to determine an amount of cell-free DNA from a non-subject source, such as DNA specific to a donor or donor-specific cell-free DNA (e.g., donor-specific cf-DNA) or fetal DNA (e.g., fetal cell-free DNA). Any one of the methods or systems provided herein may be used on a sample from a subject that has undergone a transplant. In some embodiments, the transplant is a heart transplant. Any one of the methods or systems provided herein may be used on a sample from a pregnant subject.

“Cell-free DNA” (cf-DNA) refers to fragments of DNA that are the released from cells, without wishing to be bound by any theory, generally during apoptosis, lysis, necrosis, or injury which are found freely circulating, e.g., in the blood, plasma, serum, urine, etc. of a subject, As used herein, the compositions and methods provided herein can be used to determine an amount of cell-free DNA, for example non-subject cell-free DNA, such as of a donor that can be found in a transplant recipient or such as of a pregnant subject. “Subject” cf-DNA can be uniquely quantified and detected as distinct from “non-subject” cf-DNA, such as in the case of transplant subjects or fetal DNA in maternal serum during pregnancy (Norton et. al., N Engl J Med 373: 2582 (2015)).

The systems and methods provided herein can employ the use of simulations, such as Monte Carlo simulations, when the non-subject genotype is not known. Generally, the systems and methods analyze amounts of alleles at a number of targets. A “target” is a nucleic acid sequence within which there is, may be or there is an expectation of sequence identity variability. In an embodiment, the target is, may be or is expected to be one where there is sequence variability at a single nucleotide, such as in a population of individuals or as a result of a mutation that can occur in a subject and that can be associated with a disease or condition. The target, thus, has or is expected to have more than one allele, and in preferred embodiments, the target is biallelic. A “plurality of targets” refers to more than one target (i.e., multiple laigels).

In some embodiments of any one of the systems or methods provided, amounts of alleles are analyzed at at least 25, 30, 35, 40, 45, 50, 55, 60, 65, 70, 75, 80, 85, 90, 91, 92, 93, 94, 95 or more targets. In some embodiments of any one of the methods or systems provided herein, amounts of alleles are analyzed at fewer than 105, 104, 103, 102, 101,100, 99, 98 or 97 targets. In some embodiments of any one of the methods or systems provided herein, amounts of alleles are analyzed for between 40-105, 45-105, 50-105, 55-105, 60-105, 65-105, 70-105, 75-105, 80-105, 85-105, 90-105, 90-104, 90-103, 90-102, 90-101, 90-100, 90-99, 91-99, 92-99, 93, 99, 94-99, 95-99, or 90-95 targets. In some embodiments of any one of the methods or systems provided, for between 40-99, 45-99, 50-99, 55-99, 60-99, 65-99, 70-99, 75-99, 80-99, 85-99, 90-99, 90-99, 90-98, 90-97 or 90-96 targets. In still other embodiments of any one of the methods or systems provided, for between 40-95, 45-95, 50-95, 55-95, 60-95, 65-95, 70-95, 75-95, 80-95, 85-95, or 90-95 targets. In still other embodiments of any one of the methods or systems provided, for between 40-90, 45-90, 50-90, 55-90, 60-90, 65-90, 70-90, 75-90, 80-90, or 85-90 targets. In still other embodiments of any one of the methods or systems provided, for between 40-85, 45-85, 50-85, 55-85, 60-85, 65-85, 70-85, 75-85, or 80-85 targets. In still other embodiments of any one of the methods or systems provided, for between 40-80, 45-80, 50-80, 55-80, 60-80, 65-80, 70-80, or 75-80 targets. In still other embodiments of any one of the methods or systems provided, for between 40-75, 45-75, 50-75, 55-75, 60-75, 65-75, or 70-75 targets.

Targets may be identified as quantifiable (i.e., that an allele amount can be measured) and/or informative. “Informative targets” as provided herein are those where amounts of the alleles can be used to quantify an amount of non-subject nucleic acids relative to or distinguished from subject nucleic acids in a sample. Generally, informative results can exclude the results where the subject nucleic acids are heterozygous for a specific target as well as “no call” or erroneous call results. From the informative results, allele amounts (e.g., ratios or percentages) can be calculated, such as using standard curves, in some embodiments of any one of the methods or systems provided. In some embodiments of any one of the methods or systems provided, the amount of non-subject and/or subject nucleic acids represents an average across informative results for the non-subject and/or subject nucleic acids, respectively. In some embodiments of any one of the methods or systems provided herein, this average is given as an absolute amount or as a ratio or percentage. Preferably, in some embodiments of any one of the methods or systems provided herein, this average is a mean or the median. In other embodiments of any one of the methods or systems provided herein, the average is a trimmed mean. As used herein, the “trimmed mean” refers to the removal of the lowest reporting targets (such as the two lowest) in combination with the highest of the reporting targets (such as the two highest). In still other embodiments of any one of the methods or systems provided herein, the average is the mean.

In another aspect are reports of any one of more of the values produced using any one of the methods or systems provided herein. In one embodiment, the report provides an amount of non-subject cell-free DNA at one or more time points. In one embodiment, the report can include and/or can also include any one or more other values produced by any one of the methods or systems provided herein. Preferably, a report is one in which at least one of the values can be used by a clinician for assessing the subject and/or treating a subject. Any one or more of the methods provided herein can include a step of generating a report and/or providing a report and/or assessing a subject based on one or more values and/or treating a subject based on one or more values produced by any one of the methods or systems or provided in any one of the reports provided herein.

Reports may be in oral, written (or hard copy) or electronic form, such as in a form that can be visualized or displayed. In some embodiments, the “raw” results provided herein are provided in a report, and from this report, further steps can be taken to determine the amount of non-subject nucleic acids in the sample. In other embodiments, the report provides the amount of non-subject nucleic acids in the sample. From the amount, in some embodiments, a clinician may assess the need for a treatment for the subject or the need to monitor the subject, such as the amount of the non-subject nucleic acids later in time. Accordingly, in any one of the methods provided herein, the method can include assessing the amount of non-subject nucleic acids in the subject at another point in time. Such assessing can be performed with any one of the methods provided herein. In some embodiments, the report provides amounts of non-subject nucleic acids from a subject over time.

In some embodiments of any one of the methods or systems provided herein, the amounts are in or entered into a database. In one aspect, a database with such values is provided. From the amount(s), a clinician may assess the need for a treatment or monitoring of a subject. Accordingly, in any one of the methods provided herein, the method can include assessing the amounts in the subject at more than one point in time. Such assessing can be performed with any one of the methods or systems provided herein.

As used herein, “amount” refers to any quantitative value for the measurement of nucleic acids (e.g., cf-DNA) and can be given in an absolute or relative amount. Further, the amount can be a total amount, frequency, ratio, percentage, etc. As used herein, the term “level” can be used instead of “amount” but is intended to refer to the same types of values. Generally, unless otherwise provided, the amounts provided herein represent the ratio or percentage of non-subject nucleic acids in a sample.

In some embodiments, any one of the methods or systems provided herein can comprise an analytic component configured to compare an amount to a threshold value, or to one or more prior amounts, to identify a subject at increased or decreased risk. For example, the analytic component can simulate a donor genotype to enable analysis of a mixed genotype sample where the non-subject genotype is unknown. In another example, the analytic component is configured to compare a value obtained (reflective of an amount of non-subject (e.g., donor) nucleic acids (e.g., cf-DNA)) in a sample against target threshold for increased risk. Where a measurement or value falls below the thresholds the subject can be labeled low risk or in some instance not increased risk, and where the values exceed the threshold the subject can he identified as increased risk. The analytic component can also compare the measurement or value against thresholds for reduced risk. If the subject is below the thresholds, the subject can be identified as low risk. If not the subject can received no label or also be evaluated against high risk thresholds.

“Threshold” or “threshold value”, as used herein, refers to any predetermined level or range of levels that is indicative of something. For example, in determining risk this threshold can be of the presence or absence of a condition or the presence or absence of a risk. The threshold value can take a variety of forms. It can be single cut-off value, such as a median or mean. It can be established based upon comparative groups, such as where the risk in one defined group is double the risk in another defined group. It can be a range, for example, where the tested population is divided equally (or unequally) into groups, such as a low-risk group, a medium-risk group and a high-risk group, or into quadrants, the lowest quadrant being subjects with the lowest risk and the highest quadrant being subjects with the highest risk. The threshold value can depend upon the particular population selected or the purpose of the value that is being measured and compared to a threshold. Appropriate values, ranges and categories of thresholds can be selected with no more than routine experimentation by those of ordinary skill in the art.

Because of the ability to determine amounts of non-subject nucleic acids, even at low levels, the methods and systems provided herein can be used to assess a risk in a subject, such as a transplant recipient or pregnant subject. A “risk” as provided herein, refers to the presence or absence of any undesirable condition in a subject (such as a transplant recipient), or an increased likelihood of the presence or absence of such a condition, e.g., transplant rejection. As provided herein “increased risk” refers to the presence of any undesirable condition in a subject or an increased likelihood of the presence of such a condition. As provided herein, “decreased risk” refers to the absence of any undesirable condition in a subject or a decreased likelihood of the presence (or increased likelihood of the absence) of such a condition. In some embodiments of any one of the methods provided herein, a subject having an increased amount compared to a threshold value, or to one or more prior amounts, is identified as being at increased risk. In some embodiments of any one of the methods provided herein, a subject having a decreased or similar amount compared to a threshold value, or to one or more prior amounts, is identified as being at decreased or not increased risk.

As an example, early detection of rejection following implantation of a transplant (e.g., a heart transplant) can facilitate treatment and improve clinical outcomes. Transplant rejection remains a major cause of graft failure and late mortality and generally requires lifelong surveillance monitoring. Treatment of transplant rejections with immunosuppressive therapy has been shown to improve treatment outcomes, particularly if rejection is detected early. A clinician can make an assessment (e.g., assessing the risk) of a transplant subject with an amount of donor cf-DNA and such a step can be included as part of any one of the methods provided herein.

Accordingly, in some embodiments of any one of the methods or systems provided, the subject is a recipient of a transplant, and the risk is a risk associated with the transplant. In some embodiments of any one of the methods or systems provided, the risk associated with the transplant is risk of transplant rejection, an anatomical problem with the transplant or injury to the transplant. In some embodiments of any one of the methods or systems provided, the injury to the transplant is initial or ongoing injury. In some embodiments of any one of the methods or systems provided, the risk associated with the transplant is an acute condition or a chronic condition. In some embodiments of any one of the methods or systems provided, the acute condition is transplant rejection including cellular rejection or antibody mediated rejection. In some embodiments of any one of the methods or systems provided, the chronic condition is graft vasculopathy. In some embodiments of any one of the methods or systems provided, the risk associated with the transplant is indicative of the severity of the injury. In some embodiments of any one of the methods or systems provided, the risk associated with the transplant is risk or status of an infection. The risk in a recipient of a transplant can be determined as part of any one of the methods provided herein.

As used herein, “transplant” refers to the moving of tissue or an organ or portion thereof from a donor to a recipient for the purpose of replacing the recipient's damaged or absent tissue or organ or portion thereof. The transplant may be of one organ or more than one organ. Examples of organs that can be transplanted include, but are not limited to, the heart, kidney(s), kidney, liver, lung(s), pancreas, intestine, etc. Any one of the methods or systems provided herein may be used on a sample from a subject that has undergone a transplant of any one or more of the tissues or organs, or portions thereof, provided herein. In some embodiments, the transplant is a heart transplant.

In some embodiments of any one of the methods or systems provided herein, the method or system can comprise correlating an increase in an amount of non-subject nucleic acids relative to subject or total nucleic acids with an increased risk of a condition, such as transplant rejection. In some embodiments of any one of the methods or systems provided herein, correlating comprises comparing an amount (e.g., concentration, ratio or percentage) of non-subject nucleic acids to a threshold value to identify a subject at increased or decreased risk of a condition. In some embodiments of any one of the methods or systems provided herein, a subject having an increased amount of non-subject nucleic acids compared to a threshold value is identified as being at increased risk of a condition. In some embodiments of any one of the methods or systems provided herein, a subject having a decreased or similar amount of non-subject nucleic acids compared to a threshold value is identified as being at decreased risk of a condition.

Changes in the amounts of non-subject nucleic acids can also be monitored over time, and any one of the methods or systems provided herein can include a step of doing so. This can allow for the measurement of variations in a clinical state and/or permit calculation of normal values or baseline levels. In organ transplantation, this can form the basis of an individualized non-invasive screening test for rejection or a risk of a condition associated thereto. Generally, as provided herein, the amount, such as the ratio or percent, of non-subject nucleic acids can be indicative of the presence or absence of a risk associated with a condition, such as risk associated with a transplant, such as rejection, in the recipient, or can be indicative of the need for further testing or surveillance. In one embodiment of any one of the methods or systems provided herein, the method or system may further include an additional test(s) for assessing a condition, such as transplant rejection, transplant injury, etc., or a step of suggesting such further testing to the subject (or providing information about such further testing). The additional test(s) may be any one of the methods or systems provided herein. The additional test(s) may be any one of the other methods or systems provided herein or otherwise known in the art as appropriate.

Any one of the method or systems provided herein can include a step of “determining a treatment regimen”, which refers to the determination of a course of action for the treatment of the subject. In one embodiment of any one of the methods or systems provided herein, determining a treatment regimen includes determining an appropriate therapy or information regarding an appropriate therapy to provide to a subject. In any one of the methods or systems provided herein, the determining can include providing an appropriate therapy or information regarding an appropriate therapy to a subject. In some embodiments, the therapy is administration of an anti-rejection treatment and/or anti-infection treatment. As used herein, information regarding a treatment or therapy or monitoring may be provided in written form or electronic form. In some embodiments, the information may be provided as computer-readable instructions. In some embodiments, the information may be provided orally.

“Administering” or “administration” or “administer” or the like means providing a material to a subject in a manner that is pharmacologically useful directly or indirectly. Thus, the term includes directing, such as prescribing, the subject or another party to administer the material. Administration of a treatment or therapy may be accomplished by any method known in the art (see, e.g., Harrison's Principle of Internal Medicine, McGraw Hill Inc.). Preferably, administration of a treatment or therapy occurs in a therapeutically effective amount. Administration may be local or systemic. Administration may be parenteral (e.g., intravenous, subcutaneous, or intradermal) or oral. Compositions for different routes of administration are known in the art (see, e.g., Remington's Pharmaceutical Sciences by E. W. Martin).

In some embodiments, the anti-rejection treatment administered is an immunosuppressive. Immunosuppressives include, but are not limited to, corticosteroids (e.g., prednisolone or hydrocortisone), glucocorticoids, cytostatics, alkylating agents (e.g., nitrogen mustards (cyclophosphamide), nitrosoureas, platinum compounds, cyclophosphamide (Cytoxan)), antimetabolites (e.g., folic acid analogues, such as methotrexate, purine analogues, such as azathioprine and mercaptopurine, pyrimidine analogues, and protein synthesis inhibitors), cytotoxic antibiotics (e.g., dactinomycin, anthracyclines, mitomycin C, bleomycin, mithramycin), antibodies (e.g., anti-CD20, anti-IL-1, anti-IL-2Ralpha, anti-T-cell or anti-CD-3 monoclonals and polyclonals, such as Atgam, and Thymoglobuline), drugs acting on immunophilins, ciclosporin, tacrolimus, sirolimus, interferons, opiods, TNF-binding proteins, mycophenolate, fingolimod and myriocin. In some embodiments, anti-rejection therapy comprises blood transfer or marrow transplant. Therapies can also include therapies for treating systemic conditions, such as sepsis. The therapy for sepsis can include intravenous fluids, antibiotics, surgical drainage, early goal directed therapy (EGDT), vasopressors, steroids, activated protein C, drotrecogin alfa (activated), oxygen and appropriate support for organ dysfunction. This may include hemodialysis in kidney failure, mechanical ventilation in pulmonary dysfunction, transfusion of blood products, and drug and fluid therapy for circulatory failure. Ensuring adequate nutrition—preferably by enteral feeding, but if necessary by parenteral nutrition—can also be included particularly during prolonged illness. Other associated therapies can include insulin and medication to prevent deep vein thrombosis and gastric ulcers.

In some embodiments, wherein infection is indicated, therapies for treating a recipient of a transplant can also include therapies for treating a bacterial, fungal and/or viral infection. Such therapies include antibiotics. Other examples include, but are not limited to, amebicides, aminoglycosides, anthelmintics, antifungals, azole antifungals, echinocandins, polyenes, diarylquinolines, hydrazide derivatives, nicotinic acid derivatives, rifamycin derivatives, streptomyces derivatives, antiviral agents, chemokine receptor antagonist, integrase strand transfer inhibitor, neuraminidase inhibitors, NNRTIs, NS5A inhibitors, nucleoside reverse transcriptase inhibitors (NRTIs), protease inhibitors, purine nucleosides, carbapenems, cephalosporins, glycylcyclines, leprostatics, lincomycin derivatives, macrolide derivatives, ketolides, macrolides, oxazolidinone antibiotics, penicillins, beta-lactamase inhibitors, quinolones, sulfonamides, and tetracyclines. Other such therapies are known to those of ordinary skill in the art. Any one of the methods provided herein can include administering or suggesting an anti-infection treatment to the subject (including providing information about the treatment to the subject, in some embodiments). In some embodiments, an anti-infection treatment may be a reduction in the amount or frequency in an immunosuppressive therapy or a change in the immunosuppressive therapy that is administered to the subject. Other therapies are known to those of ordinary skill in the art.

Any one of the method or systems provided herein can include a step of “determining a monitoring regimen”, which refers to determining a course of action to monitor a condition in the subject over time. In one embodiment of any one of the methods or systems provided herein, determining a monitoring regimen includes determining an appropriate course of action for determining the amount of non-subject nucleic acids in the subject over time or at a subsequent point in time, or suggesting such monitoring to the subject. This can allow for the measurement of variations in a clinical state and/or permit calculation of normal values or baseline levels (as well as comparisons thereto). In some embodiments of any one of the methods or systems provided herein determining a monitoring regimen includes determining the timing and/or frequency of obtaining samples from the subject.

As used herein, the sample from a subject can be a biological sample. Examples of such biological samples include whole blood, plasma, serum, urine, etc. In some embodiments of any one of the methods provided herein, addition of further nucleic acids, e.g., a standard, to the sample can be performed.

In any one of the methods or systems provided herein, amounts of alleles can be determined with sequencing, such as a next generation or high-throughput sequencing and/or genotyping technique. Examples of next generation and high-throughput sequencing and/or genotyping techniques include, but are not limited to, massively parallel signature sequencing, polony sequencing, 454 pyrosequencing, Illumina (Solexa) sequencing, SOLiD sequencing, ion semiconductor sequencing, DNA nanoball sequencing, heliscope single molecule sequencing, single molecule real time (SMRT) sequencing, MassARRAY®, and Digital Analysis of Selected Regions (DANSR™) (see, e.g., Stein R A (1 Sep. 2008). “Next-Generation Sequencing Update”. Genetic Engineering & Biotechnology News 28 (15); Quail, Michael; Smith, Miriam E; Coupland, Paul; Otto, Thomas D; Harris, Simon R; Connor, Thomas R; Bertoni, Anna; Swerdlow, Harold P; Gu, Yong (1 Jan. 2012). “A tale of three next generation sequencing platforms: comparison of Ion torrent, pacific biosciences and illumina MiSeq sequencers”. BMC Genomics 13 (1): 341; Liu, Lin; Li, Yinhu; Li, Siliang; Hu, Ni; He, Yimin; Pong, Ray; Lin, Danni; Lu, Lihua; Law, Maggie (1 Jan. 2012). “Comparison of Next-Generation Sequencing Systems”. Journal of Biomedicine and Biotechnology 2012: 1-11; Qualitative and quantitative genotyping using single base primer extension coupled with matrix-assisted laser desorption/ionization time-of-flight mass spectrometry (MassARRAY®). Methods Mol Biol. 2009; 578:307-43; Chu T, Bunce K, Hogge W A, Peters D G. A novel approach toward the challenge of accurately quantifying fetal DNA in maternal plasma. Prenat Diagn 2010; 30:1226-9; and Suzuki N, Kamataki A, Yamaki J, Homma Y. Characterization of circulating DNA in healthy human plasma. Clinica chimica acta; International Journal of Clinical Chemistry 2008; 387:55-8). Such methods may also be used to determine genotype in some embodiments.

In any one of the methods or systems provided herein, amounts of alleles can be determined with an amplification technique, such a method as described herein or in U.S. Publication No. WO 2016/176662. Any one of such techniques are incorporated herein.

In some embodiments of any one of the methods provided herein, the amplification is performed with PCR, such as quantitative PCR meaning that amounts of nucleic acids can be determined. Quantitative PCR include real-time PCR, digital PCR, TAQMAN™, etc. In some embodiments of any one of the methods or systems provided herein the PCR is “real-time PCR”. Such PCR refers to a PCR reaction where the reaction kinetics can be monitored in the liquid phase while the amplification process is still proceeding. In contrast to conventional PCR, real-time PCR offers the ability to simultaneously detect or quantify in an amplification reaction in real time. Based on the increase of the fluorescence intensity from a specific dye, the concentration of the target can be determined even before the amplification reaches its plateau. In some embodiments of any one of the methods provided, the PCR is digital PCR.

### System Implementation

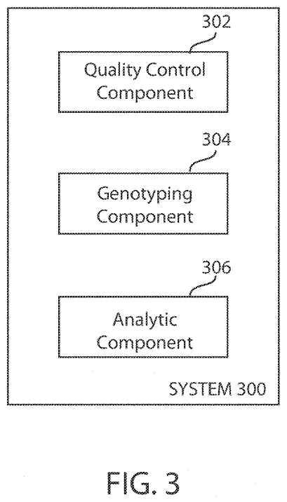

According to one aspect, a system is provided for calculating quality measures on a sample taken from a subject, such as a transplant recipient. Various embodiments of any one of the systems are configured to identify samples having higher or lower risk properties responsive to analyzing genomic data obtained from a subject. FIG. 3 illustrates one example system 300 for identifying such samples and risk profile. According to one embodiment of any one of the systems, the system can be configured to analyze the sample directly or data regarding the sample to provide “quantitative genotyping” (qGT). According to some embodiments of any one of the systems, the system executes quantitative genotyping that uses standard curves of heterozygous DNA sources to quantify the A and B alleles at each target. Further embodiments of any one of the systems execute quality control procedures to evaluate each standard curve and sample amplification according to acceptability criteria. According to some embodiments of any one of the systems, the system can be configured to classify data that meets the quality control procedures as quantifiable targets, and execute interpretation algorithms on the quality controlled data.

According to some embodiments of any one of the systems, quality control is based on specific acceptability criteria which can include analysis of any one or more and any combination of the following: historic amplification shape, specificity of the allele specific PCR assay with respect to the second allele, Cp or Ct values, PCR efficiency, signal to noise, slope and r-squared of standard curve sets, non-amplification of controls, or contamination of negative controls.

According to one embodiment of any one of the systems, the system includes a quality control component 302 that executes the analysis and/or disclosed algorithms for identifying quantifiable targets.

According to some embodiments of any one of the systems, the system (e.g., 300) provides a primary analysis of genotype. For example, the system can first evaluate recipient (or subject) and donor (or non-subject) genomes for “basic genotyping” (bGT). The bGT process generates labels for the donor (or non-subject) and/or recipient (or subject) three possible genotypes at each target (e.g., homozygous AA, heterozygous AB, and homozygous BB). According to various embodiments of any one of the systems, this information is used by the system in the interpretation of the qGT per target. According to one embodiment of any one of the systems, the system 300 can include a genotyping component 304 configured to analyze genotype of a donor (or non-subject) and/or recipient (or subject) contribution to a sample at specified targets. According to one embodiment of any one of the systems, the identification of the genotype at each target allows the system to recognize informative targets, such as fully and/or half informative, based on genotype.

For example, the system can be configured to define informative targets as those where the recipient (or subject) is known homozygous and the donor (or non-subject) has another genotype. In one example, the system identifies the informative targets, stores information on respective targets that are informative and includes the labels for the donor and/or recipient and the result of analyzing genotype of both.

According to another embodiment of any one of the systems, a genotyping component (e.g., 304) labels donor (or non-subject) and/or recipient (or subject) targets to analyze the informative targets. In another example, the system is configured to identify an informative target, where the donor (or non-subject) is homozygous for the other allele (different from a homozygous recipient (or subject)). In further embodiments of any one of the systems, the genotyping component can be configured to classify respective targets as fully informative or half informative responsive to analysis of observed allele ratios.

In this example, the target is referred to as fully informative, and the observed allele ratio is approximately the overall donor cf-DNA (or non-subject cf-DNA) level. In further examples, instances are identified by the system where the donor (or non-subject) is heterozygous and the recipient (or subject) is homozygous, and the target is defined as half informative (because the contribution is to both the A and B alleles). For half informative targets, the system is configured to adjust the measured contribution. For example, responsive to determining a target is half informative the measured contribution can be doubled. In other embodiments, more refined adjustments can be executed. For example, ratios of donor cf-DNA to recipient cf-DNA can be expressed as percentages. The percent value can be used to adjust measured contributions accordingly. In other example, the adjustment to the measured contribution can be based on statistical variation, among other options.

According to various embodiments of any one of the systems, the system is configured to generate the median of informative and quality-control-passed allele ratios and output the median as the percentage of donor cell free DNA (or non-subject cf-DNA). The system can be configured to report the median of informative and quality-control-passed allele ratios and output the median as the percentage of donor cell free DNA to improve the robustness of the calculated results. In some implementations of any one of the systems, the system includes a genotyping component (e.g., 304) configured to label donor (or non-subject) and/or recipient (or subject) targets, and adjust any measured contributions as needed.

According to one embodiment of any one of the systems, the system executed qGT process generates at least two quality measures (e.g., assessment of usefulness of a value), a robust Coefficient of Variation (rCV) and a dQC. For example, the system can be configured to calculate the regularized (rCV) using the distribution of the informative and quantifiable targets.

In one approach, a robust standard deviation (rSD) is computed as the median absolute divergence from the median minor species proportion, scaled by a normalizing factor (e.g., of 1.4826). The rSD can be converted to a coefficient of variation by dividing by the donor cf-DNA % (or non-subject cf-DNA %) after it has been regularized by adding a stub value (e.g., a quarter of one percent). The stub value can be introduced by the system to avoid instability around a zero divisor, and includes in various examples, a small value to ensure a non-zero divisor. In various embodiments, the system can be configured to measure the spread of assayed targets around their median with the rCV. This allows the system to determine the rCV as a metric of precision or sample quality. The system can be configured to apply the sample quality metric to identify health samples. In some examples, useful samples can have a rCV below 50%. The result of the improved quality metrics yields increases in sample anomalies detection, as well as improvement in adverse condition detection over conventional approaches.

According to one embodiment of any one of the systems, the system 300 can include an analytic component 306 configured to calculate various quality measures on sample data (including for example adjusted sample data based on genotype). In one example, the analytic component is configured to calculate rSD, rCV, and dQC to ensure sample stability and ensure no contamination of the sample has occurred.

According to some embodiments of any one of the systems, the system determines the dQC value to provide a discordance quality check: the system is configured to evaluate the average minor allele proportion of recipient homozygous and non-informative targets as a safeguard against sample mix-ups and contamination. “dQC” values should theoretically read nearly zero percent, subject to non-specificity allelic noise. If a sample-swap had occurred during collection or processing, the wrong recipient genotypes are used, and the dQC test executed by the system immediately flags up to 50 or 100% readings at presumed non-informative targets. Further embodiments of any one of the systems, implement dQC analysis to identify sample contamination and genomic instability in the sample. The system can be set with a default value to identify data as useful samples when a calculated dQC value falls below, for example, 0.5%. Other thresholds can be implemented (e.g., <1%, 2%, 0.3%, 0.4%, 0.6%, etc.). Further example thresholds include 1%, 5%, 10%, or 50%. In various embodiments of any one of the systems, execution of dQC filtering improves detection of contamination and/or detection of genomic instability over conventional approaches.

In a further aspect (or in further embodiments of any one of the other systems provided), a system configured with methods to simulate donor (or non-subject) genotype and then, in some embodiments, calculate donor cf-DNA(or non-subject cf-DNA), is provided (or can be so configured). For example, if a donor (or non-subject) genotype is not available the system can still calculate donor cf-DNA (or non-subject cf-DNA) based on simulation of donor (or non-subject) genotype data. Simulating donor (or non-subject) genotype enables the system (e.g., 300) to determine probable donor (or non-subject) genotype and ranges for probable qGT outcomes. According to various embodiments of any one of the systems, the system is configured to generate wholly random genotypes and execute statistical calculations to identify the more likely non-self genotypes. The system can repeat the random genotype generation with biases applied to alleles which are evidently visible.

According to various embodiments of any one of the systems, the system (e.g., 300) is configured to execute a simulation method to compute donor cf-DNA (or non-subject cf-DNA) when the donor genotype is not available. Using just the recipient's genotypes and qGT results, the system evaluates donor (or non-subject) options using a Monte Carlo simulation. For example, the preliminary random selections in the simulations determine what overall results a given qGT sample could represent. The statistical analyses of the simulation findings by the system establish probable donor (or non-subject) genotypes. The system can also be configured to execute secondary Monte Carlo simulations to explore the likely donor (or non-subject) genotype space and yield a range of probable qGT outcomes. According to one example, each of fifty thousand simulations executed by the system reports a median donor cf-DNA (or non-subject cf-DNA), rCV and dQC triplet, creating a three dimensional point cloud. In subsequent processing on the system, the point cloud is sliced for the lower-third of dQC and rCV and the remaining “quadrant” represents the simulations corresponding to a realistic and clean sample. The central 95% of the donor cf-DNA (or non-subject cf-DNA) calls can yield a “Method 2” outcome for the qGT without having donor (or non-subject) genotype, in some embodiments. In other implementations, fewer simulations (e.g., ten thousand, twenty thousand, thirty thousand, etc.) can be executed or a larger number of simulations (e.g., sixty thousand, seventy thousand, etc.) can be executed to establish values for processing. According to some embodiments of any one of the systems, additional calculations can be applied to refine genotype simulations and resulting predictions of donor genotype.

Various aspects and functions described herein (e.g., execution of basic genotyping algorithms, specific genotyping algorithms, qGT algorithms, manipulation of sample recorded data to transform the sample results (e.g., into genotypic normalized appearance values), “without donor (or non-subject)” algorithms, (re)simulation algorithms, Monte-Carlo simulations, etc.), may be implemented as specialized hardware or software components executing in one or more specially configured computer systems (e.g., network appliances, personal computers, workstations, mainframes, networked clients, servers, media servers, application servers, database servers, web servers, mobile computing devices (e.g., smart phones, tablet computers, and personal digital assistants) and network equipment (e.g., load balancers, routers, and switches)). Further, aspects may be located on a single computer system or may be distributed among a plurality of computer systems connected to one or more communications networks.

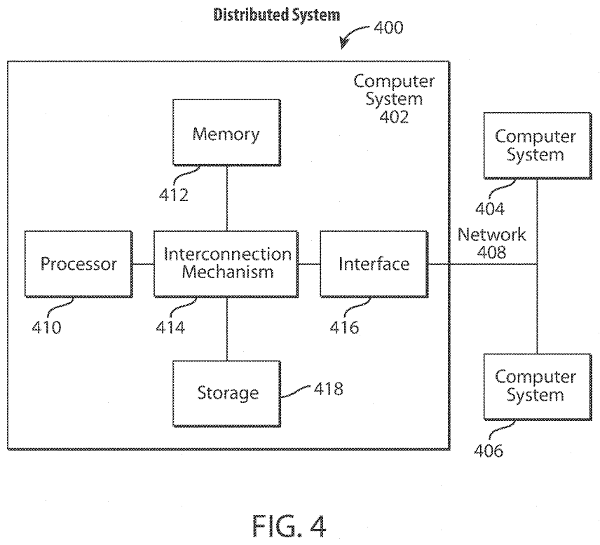

For example, various aspects, functions, system components, and processes (e.g., quality control component, genotyping component, and analytic component) may be located on singular computer systems or distributed among one or more computer systems (including cloud resources) specially configured to provide a service to one or more client computers, or to specially configured to perform an overall task as part of a distributed system, such as the distributed computer system 400 shown in FIG. 4. Consequently, embodiments are not limited to executing on any particular system or group of systems. Further, aspects, functions, and processes may be implemented in software, hardware or firmware, or any combination thereof. According to some embodiments of any one of the systems, computer system 400 can be connected to other systems for processing tissue and/or blood samples to yield cf-DNA values or to analyze the values capture from the same to determine sample quality, c.nntaminatinn, health and/or viahility, among other options.

Referring to FIG. 4, there is illustrated a block diagram of a special purpose distributed computer system 400, in which various aspects and functions of the disclosure are practiced. As shown, the distributed computer system 400 includes one or more computer systems that exchange information. More specifically, the distributed computer system 400 includes computer systems 402, 404, and 406. As shown, the computer systems 402, 404, and 406 are interconnected by, and may exchange data through, a communication network 408. The network 408 may include any communication network through which computer systems may exchange data. To exchange data using the network 408, the computer systems 402, 404, and 406 and the network 408 may use various methods, protocols and standards, including, among others, Fiber Channel, Token Ring, Ethernet, Wireless Ethernet, Bluetooth, IP, IPV6, TCP/IP, UDP, DTN, HTTP, FTP, SNMP, SMS, MMS, SS4, JSON, SOAP, CORBA, REST, and Web Services. To ensure data transfer is secure, the computer systems 402, 404, and 406 may transmit data via the network 408 using a variety of security measures including, for example, SSL or VPN technologies. While the distributed computer system 400 illustrates three networked computer systems, the distributed computer system 400 is not so limited and may include any number of computer systems and computing devices, networked using any medium and communication protocol.

As illustrated in FIG. 4, the computer system 402 includes a processor 410, a memory 412, an interconnection element 414, an interface 416 and data storage element 418. To implement at least some of the aspects, functions, and processes disclosed herein, the processor 410 performs a series of instructions that result in manipulated data. The processor 410 may be any type of processor, multiprocessor or controller. Example processors may include a commercially available processor. The processor 410 is connected to other system components, including one or more memory devices 412, by the interconnection element 414.

The memory 412 stores programs (e.g., sequences of instructions coded to be executable by the processor 410) and data during operation of the computer system 402. Thus, the memory 412 may be a relatively high performance, volatile, random access memory such as a dynamic random access memory (“DRAM”) or static memory (“SRAM”). However, the memory 412 may include any device for storing data, such as a disk drive or other nonvolatile storage device. Various examples may organize the memory 412 into particularized and, in some cases, unique structures to perform the functions disclosed herein. These data structures may be sized and organized to store values for particular data and types of data.

Components of the computer system 402 are coupled by an interconnection element such as the interconnection element 414. The interconnection element 414 may include any communication coupling between system components such as one or more physical busses in conformance with specialized or standard computing bus technologies. The interconnection element 414 enables communications, including instructions and data, to be exchanged between system components of the computer system 402.

The computer system 402 also includes one or more interface devices 416 such as input devices, output devices and combination input/output devices. Interface devices may receive input or provide output. More particularly, output devices may render information for external presentation. Input devices may accept information from external sources. Examples of interface devices include keyboards, mouse devices, trackballs, microphones, touch screens, printing devices, display screens, speakers, network interface cards, etc. Interface devices allow the computer system 402 to exchange information and to communicate with external entities, such as users and other systems.

The data storage element 418 includes a computer readable and writeable nonvolatile, or non-transitory, data storage medium in which instructions are stored that define a program or other object that is executed by the processor 410. The data storage element 418 also may include information that is recorded, on or in, the medium, and that is processed by the processor 410 during execution of the program. The instructions may be persistently stored as encoded signals, and the instructions may cause the processor 410 to perform any of the functions described herein. The medium may, for example, be optical disk, magnetic disk or flash memory, among others. In operation, the processor 410 or some other controller causes data to be read from the nonvolatile recording medium into another memory, such as the memory 412, that allows for faster access to the information by the processor 410 than does the storage medium included in the data storage element 418. The memory may be located in the data storage element 418 or in the memory 412, however, the processor 410 manipulates the data within the memory, and then copies the data to the storage medium associated with the data storage element 418 after processing is completed. A variety of components may manage data movement between the storage medium and other memory elements and examples are not limited to particular data management components. Further, examples are not limited to a particular memory system or data storage system.

Although the computer system 402 is shown by way of example as one type of computer system upon which various aspects and functions may be practiced, aspects and functions are not limited to being implemented on the computer system 402 as shown in FIG. 4. Various aspects and functions may be practiced on one or more computers having a different architectures or components than that shown in FIG. 4.

The computer system 402 may be a computer system including an operating system that manages at least a portion of the hardware elements included in the computer system 402. The processor 410 and operating system can together define a computer platform for which application programs in high-level programming languages are written. Additionally, various aspects and functions may be implemented in a non-programmed environment. For example, documents created in HTML, XML or other formats, when viewed in a window of a browser program, can render aspects of a graphical-user interface or perform other functions. Further, various examples may be implemented as programmed or non-programmed elements, or any combination thereof.

### EXAMPLE

A total of 298 samples from 87 unique transplant recipient subjects both adult and pediatric passed quality control (QC) standards and were available for analysis. One individual participated in the study both after initial transplantation and after retransplantation and was analyzed as two unique subjects, given the two unique donor/recipient mismatched DNA. The mean patient age at transplant was 7.9+/−7.5 years (range 0.03 to 24.2 years); the mean age at blood sample was 12.7+/−8.1 years (range 0.08 to 30.2 years); 59.6% (51/87) of the subjects were male, and 65.5% (57/87) were white. The mean time from transplant to blood sample was 4.8+/−4.2 years.

### Correlation Between Donor Fraction and Cellular Rejection Grade in Biopsy-Associated Blood Samples

A total of 158 samples were taken within 24 hours prior to EMB and included for analysis. Only one sample was associated with each biopsy. Results are summarized in Table 1. 134 biopsies were grade CR0, 21 biopsies were grade CR1, and 3 biopsies were grade CR2.

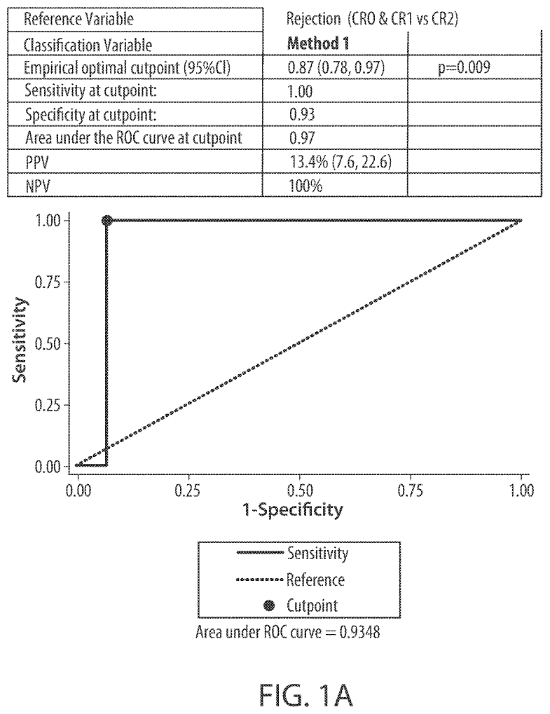

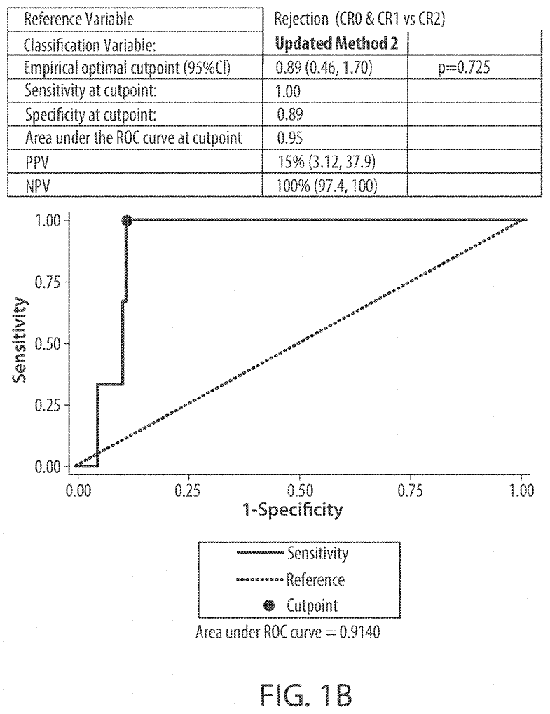

When the donor genotype was known for the analysis, the mean donor cf-DNA fraction was found to be 0.11% (IQR 0.06-0.21%) in samples associated with grade CR0 biopsies, 0.37% (IQR 0.15-0.72%) in samples associated with grade CR1 biopsies, and 0.97% (IQR 0.88-1.06%) in samples associated with grade CR2 biopsies (p=0.027). The empirical optimal cutpoint for ruling out grade CR2 rejection based on the associated ROC curve was 0.87% [95% CI 0.78-0.97% (p=0.009)]. The PPV was 13.4% (7.6, 22.6) and the NPV was 100%. A graphical representation of the data is presented in FIG. 1A.

When the donor genotype was unknown, the mean donor cf-DNA fraction was 0.25% (IQR 0.17-0.39%) in samples associated with grade CR0 biopsies, 0.89% (IQR 0.44-5.35%) in samples associated with grade CR1 biopsies, and 1.22% (IQR 1.04-5.18%) in samples associated with grade CR2 biopsies (p<0.001). The empirical optimal cutpoint for ruling out grade CR2 rejection based on the associated ROC curve was 0.89% [95% CI 0.46-1.70% (p=0.725)]. The PPV was 15% (3.21-37.9) and NPV was 100% (97.4, 100). A graphical representation of the data is presented in FIG. 1B.

TABLE 1
Donor Fraction and Cellular Rejection Grade

| | Rejection Grade | | | | |
|---|---|---|---|---|---|
| | CR0 | CR1 | CR2 | Null | |
| | Median [IQR] | Median [IQR] | Median [IQR] | Hypothesis* | Statistical Test |
| N | 134 | 21 | 3 | | |
| With Donor | 0.11 [0.06, 0.21] | 0.37 [0.15, 0.72] | 0.97 [0.88, 1.06] | p = 0.027 | Independent samples |
| Genotype | | | | | median test |
| Without Donor | | | | | |
| Genotype | | | | | |
| Average | 0.25 [0.17, 0.39] | 0.89 [0.44, 5.35] | 1.22 [1.04, 5.18] | p < 0.001 | Independent samples |
| | | | | | median test |

*Null hypothesis: the medians are the same across rejection grade categories (CR0 vs. CR1 vs. CR2)

Correlation with Quilty Lesions

139 samples were associated with biopsies reported for presence or absence of Quilty lesions (121 no, 18 yes). Correlations between donor cf-DNA fraction are summarized in Table 2.

When the donor genotype was known for the analysis, the mean donor cf-DNA fraction was 0.12% (IQR 0.07-0.32%) in samples associated with biopsies negative for Quilty lesions and 0.10% (IQR 0.06-0.19%) in samples associated with biopsies positive for Quilty lesions (p=0.738).

When the donor genotype was unknown, the mean donor cf-DNA fraction was 0.28% (IQR 0.18-0.53%) in samples associated with biopsies negative for Quilty lesions and was 0.21% (IQR 0.15-0.27%) in samples associated with biopsies positive for Quilty lesions (p=0.03).

TABLE 2
Donor Fraction and Presence of Quilty Lesions

| | Quilty Lesions | | | |
|---|---|---|---|---|
| | No | Yes | Null | |
| | Median [IQR] | Median [IQR] | Hypothesis* | Statistical Test |
| N | 121 | 18 | | |
| With Donor | 0.12 [0.07, 0.32] | 0.10 [0.06, 0.19] | p = 0.738 | Independent samples |
| Genotype | | | | median test |
| Without Donor | | | | |
| Genotype | | | | |
| Average | 0.28 [0.18, 0.53] | 0.21 [0.15, 0.27] | p = 0.03 | Independent samples |
| | | | | median test |

*Null hypothesis: the medians are the same across presence/absence of guilty lesions (no vs. yes)

Correlation with Coronary Artery Graft Vasculopathy (CAV)

116 blood samples were collected within 24 hours prior to selective coronary angiography. Of these, 11 demonstrated graft vasculopathy as defined by the 2010 ISHLT grading system (Mehra et al., J Heart Lung Transplant 29, 717-727 (2010)), and 99 showed no graft vasculopathy. A comparison of donor cf-DNA fractions among angiography-associated samples is summarized in Table 3.

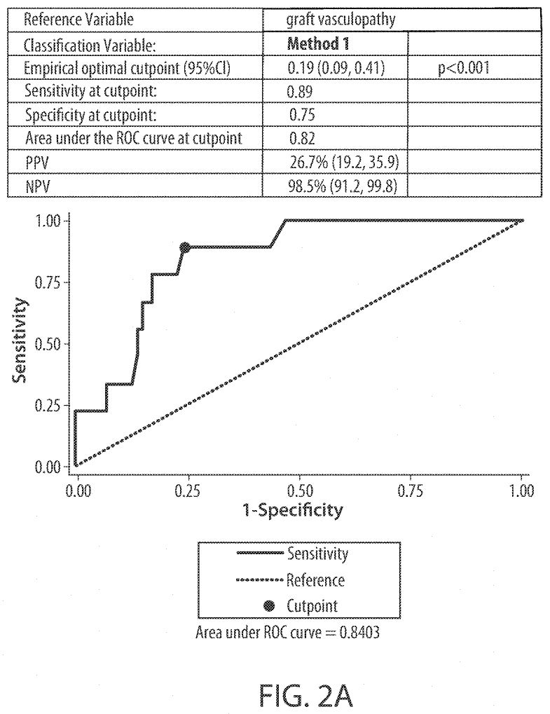

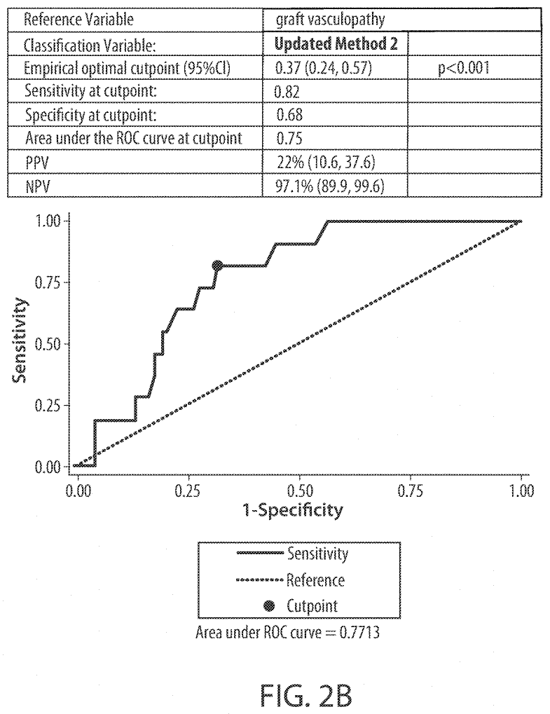

When the donor genotype was known for the analysis, the mean donor fraction was 0.09% (IQR 0.06-0.20%) for samples not associated with CAV and 0.47% (IQR 0.27-0.71%) for samples associated with CAV (p=0.05). Mehra, M. R., et al. International Society for Heart and Lung Transplantation working formulation of a standardized nomenclature for cardiac allograft vasculopathy-2010. J Heart Lung Transplant 29, 717-727 (2010). The empirical optimal cutpoint for ruling out CAV was 0.19% [95% CI 0.09-0.38% (p<0.001)]. A graphical representation of the data is presented in FIG. 2A.

When the donor genotype was unknown for the analysis, the mean donor fraction was 0.27% (IQR 0.16-0.52%) for samples not associated with CAV and 0.55% (IQR 0.38-1.22%) for samples associated with CAV (p=0.057). The empirical optimal cutpoint for ruling out CAV was 0.37% [95% CI 0.24-0.57% (p<0.001)]. A graphical representation of the data is presented in FIG. 2B.

TABLE 3
Donor Fraction and Coronary Artery Graft Vasculopathy

| | Graft Vasculopathy | | No Biopsy or | | |
|---|---|---|---|---|---|
| | No CAD | GV | Angio | Null | |
| | Median [IQR] | Median [IQR] | Median [IQR] | Hypothesis* | Statistical Test |
| N | 99 | 11 | 155 | | |
| With Donor | 0.09 [0.06, 0.20] | 0.52 [0.33, 0.88] | 0.32 [0.14, 0.87] | p = 0.028 | Independent samples |
| Genotype | | | | | median test |
| Without Donor | | | | | |
| Genotype | | | | | |
| Average | 0.27 [0.16, 0.54] | 0.55 [0.38, 1.22] | 0.057 | p = 0.057 | Independent samples |
| | | | | | median test |

*Null hypothesis: the medians are the same across no CAD and GV (no CAD vs. GV)

Correlation with Antibody-Mediated Rejection (AMR)

142 samples were associated with biopsies analyzed for antibody-mediated rejection (AMR). 132 samples were read as pAMR0 and 3 were read as grade pAMR 1 or 2. A comparison of donor cf-DNA fractions among AMR samples is summarized in Table 4.

When the donor genotype was known for the analysis, the mean donor fraction was 0.12% (IQR 0.07-0.29%) for samples associated with grade pAMR0 and was 0.26% (IQR 0.09-0.33%) for samples associated with grade pAMR1 or 2 (p=0.905).

When the donor genotype was unknown for the analysis, the mean donor fraction was 0.29% (IQR 0.18-0.61%) for samples associated with grade pAMR0 and was 0.39 (IQR 0.12-0.44%) for samples associated with grade pAMR1 or 2 (p=0.969). The empirical optimal cutpoint for ruling out pAMR1 or 2 based on the associated ROC curve was 0.38% [95% CI 0.19-0.74% (p=0.005)].

TABLE 4
Donor Fraction and Antibody-mediated Rejection

| | Antibody Mediated Rejection Grade | | | |
|---|---|---|---|---|
| | 0 | 1 or 2 | Null | |
| | Median [IQR] | Median [IQR] | Hypothesis* | Statistical Test |
| N | 132 | 3 | | |
| With Donor | 0.12 [0.07, 0.29] | 0.26 [0.09, 0.33] | p = 0.905 | Independent samples |
| Genotype | | | | median test |
| Without Donor | | | | |
| Genotype | | | | |
| Average | 0.29 [0.18, 0.61] | 0.39 [0.12, 0.44] | p = 0.969 | Independent samples |
| | | | | median test |

*Null hypothesis: the medians are the same across treatment for infection (0 vs. 1 or 2)

### Discussion

It has been found that a targeted, high-throughput assay for the quantification of donor cf-DNA has exquisite sensitivity, such as for rejection surveillance in heart transplant recipients, and that marked elevations in the donor fraction correlate to significant allograft injury, including acute episodic rejection and chronic rejection in the form of coronary artery graft vasculopathy. Specifically, the empirical optimal cutpoint of 0.87% (95% CI 0.78-0.97%) reliably distinguished CR0 and CR1 from CR2 grade rejection. The donor fraction of total cf-DNA did not distinguish between Quilty lesions, however.

Donor cf-DNA is uniquely suited as a biomarker in the field of transplantation given the genetic differences between donor and recipient. The field has progressed significantly since the first report in 1998, where the presence of a Y chromosome in the serum of female recipients was detected (Lo et al., Lancet 351: 1329-1330 (1998)).

The use of donor cf-DNA holds promise in dramatically reducing the need for surveillance biopsy and as such, allows for more frequent monitoring for rejection. Both the apparent sensitivity of the assay in detecting early rejection and the fact that it can be used at a higher frequency than EMB or other biopsies, would allow clinicians frequent non-invasive monitoring, which may result in both decreased trauma to the patient and earlier and more effective detection of rejection and/or other clinically significant events. In addition, donor cf-DNA may add to the understanding of histopathologic patterns of heart transplant recipients. The finding that patients with and without Quilty lesions had similar levels of donor cf-DNA adds to the evidence that this pathologic finding may not reflect injury to the donor organ, as others have suggested (Gopal et al., Pathol Int 48: 191-198 (1998)). Strikingly, the data showed a stepwise statistically significant difference in donor cf-DNA levels when comparing cellular grades CR0 to CR1 to CR2. This result was unexpected, and suggests a measureable linear relationship between levels of donor of DNA and progressive injury to the donor organ.

### Materials and Methods

### Measurements and Definitions

Each subject's height and weight at time of transplant and length of stay were recorded. Treatment of rejection was defined as change in immunosuppressive medications with the intent to treat allograft rejection as documented in the medical record, and initiation of treatment for rejection was recorded as the date and time this medication change was first administered to the subject. Biopsy proven cellular rejection was defined as ISHLT grade 2 or higher cellular rejection. Biopsy proven antibody-mediated rejection was defined as ISHLT grade 1 or higher AMR. Mechanical circulatory support was defined as either temporary or durable ventricular assist device, aortic balloon pump, or extra-corporeal circulatory support. If a subject was diagnosed with cancer or post-transplant lymphoproliferative disease, or became pregnant, the first dates of diagnosis were recorded, as these conditions introduce a confounding source of additional “non-self” cell-free DNA into the recipient serum. The pathology reports of all biopsies were reviewed and 2004 ISHLT grade was recorded, as well as if the biopsy was judged to have Quilty lesions. The results of coronary angiography, if performed within 24 hours prior to blood sample, were recorded according to the 2010 ISHLT grading system (Mehra et al., J Heart Lung Transplant 29: 717-727 (1998)).

Blood samples were obtained from heart transplant recipients in the following clinical scenarios: days 1, 4, 7, and 28 following transplant, within 24 hours prior to any EMB, and immediately prior to and then days 1, 4, 7, and 28 after initiation of treatment for rejection.

Mean total cf-DNA levels and interquartile ranges (IQR) were reported in ng/dL and mean percentage donor cf-DNA levels and IQRs were reported as a fraction of the total. The independent sample means test was used to compare donor fraction (percentage donor cf-DNA) and total cf-DNA (ng/ml plasma) across the clinical variables tested.

### Exclusion Criteria

In determining sensitivity and specificity of the biomarker for the pre-treatment detection of rejection, samples were excluded from analysis if the sample was collected within 8 days of cardiac transplantation, if the sample was taken within 28 days after the initiation of treatment of rejection, if the sample was taken while the patient was on mechanical circulatory support, if the subject had a diagnosis of cancer or post-transplant lymphoproliferative disease at the time of draw, or if the sample was taken after intracardiac access during the biopsy procedure, as these clinical scenarios offer biological reasons for alterations in total cf-DNA and donor fraction that confound interpretation of assay results as they relate to the early, pre-treatment, detection of rejection. Sensitivity and specificity for the diagnosis of allograft rejection was based on biopsy-associated samples that fell outside of these exclusion criteria. Subjects who were recipients of bone marrow or non-cardiac solid organ transplantation or who were pregnant prior to cardiac transplantation were also excluded from this study given that the multiple donor/recipient (and fetal) genotypes confound analysis.

Additionally, technical exclusion of samples occurred if they did not meet the following quality control (QC) standards for the assay: blood volume, plasma volume, DNA quantity, time to spin, and temperature.

### Blood Sample Collection

Three to ten milliliters (ml) of anti-coagulated blood were collected to assess circulating levels of cf-DNA. Each sample was collected in 10 ml BCT tubes (Streck, Omaha, Nebr.). Samples were immediately coded, de-identified, and delivered to the laboratory for processing.

### Plasma Processing and DNA Extraction

Separation of plasma from whole blood by centrifugation was carried out as previously described. Plasma was stored at −80° C. until DNA extraction. All cf-DNA extractions were performed using ReliaPrep™ HT Circulating Nucleic Acid Kit, Custom (Promega, Madison, Wis.). Total cf-DNA from each plasma sample was also recorded. Recipient genomic DNA was extracted by using ReliaPrep™ Large Volume gDNA Isolation system (Promega, Madison, Wis.) or Gentra Puregene Blood Kit (Qiagen, Germantown Md.). Genomic donor DNA for genotyping was obtained from the Blood Center of Southeast Wisconsin which collects and stores DNA from all donors as part of the donor/recipient matching process. In some cases, genomic DNA was obtained from biopsy samples, and extracted using a QIAamp DNA Micro Kit (Qiagen, Germantown Md.). All purified genomic DNA was re-suspended in 0.1× TE buffer.

Total cf-DNA Analysis

Total cf-DNA content in each plasma sample was evaluated in triplicate using a TaqMan quantitative real-time polymerase chain reaction (qRT-PCR) reference assay that detects the Ribonuclease P RNA component H1 (H1RNA) gene (RPPH1) on human chromosome 14, cytoband 14q11.2. The assay amplifies an 87 bp product that maps within the single exon RPPH1 gene, at chr14:20811565 on NCBI build 37 (Thermo Fisher Scientific, Waltham, Mass.). PCR analysis was carried out on an Applied Biosystems QuantStudio 7 Flex Real-Time PCR System (Thermo Fisher Scientific, Waltham, Mass.). For each reaction, one μl of cf-DNA extracted from plasma was used. A dilution series of human genomic DNA was used to create a standard curve for quantification. Total cf-DNA from each sample was obtained and presented as ng/ml of plasma.

Percentage Donor cf-DNA Analysis

A proprietary, multiplexed, allele-specific quantitative PCR-based assay called the myTAI-Heart assay was designed to directly quantify the percentage of donor cell-free DNA (Dcf-DNA) as a fraction of the total cf-DNA (TAI Diagnostics). The assay quantifies bi-allelic SNPs with real-time PCR specific to each allele. High frequency population SNPs in stable genomic regions were selected, as this increased their likelihood of reliable quantification and the discrimination ability between recipient and donor genomes.

Fifteen ng of cf-DNA was added to a multiplexed library master mixture with an exogenous standard (TAI5) spiked into each sample (4.5E+03 copies) and amplified by PCR for 35 cycles in a 25 ul reaction containing 0.005 U Q5 (NEB) DNA polymerase, 0.2 mM dNTPs, 3 uM forward primer pool of 96 targets, and 3 uM reverse primer pool of 96 targets, at a final concentration of 2 mM MgCl₂. Cycling conditions were 98° C. for 30 s, then 35 cycles of 98° C. for 10 s, 55° C. for 40 s, and 72° C. for 30 s. This was then followed by a 2 min incubation at 72° C. Samples were then stored at 4° C. Ten microliters of the final reaction was cleaned up using ExoSAP-IT(Thermo Fisher Scientific) by incubating at 37° C. for 15 minutes and the 80° C. for 15 minutes.

Samples were then diluted 1:1 with TAI preservation buffer and stored at −80° C. until ready for quantitative genotyping. Samples were then diluted 1:100 for quantitative genotyping and set up as a 3 ul reaction with appropriate controls and calibrators for a real time PCR run using a Roche LightCycler 480 system (Roche Diagnostics, Indianapolis, Ind.).

### Analysis

### Quantitative Genotyping

The “quantitative genotyping” (qGT) uses standard curves of heterozygous DNA sources to quantify the A and B alleles at each target. Quality control procedures evaluate each standard curve and sample amplification to meet acceptability criteria. Quantifiable targets are then interpreted. Acceptability criteria include historic amplification shape, specificity of the allele-specific PCR assay with respect to the second allele, signal-to-noise ratio, slope and r-squared of standard curve sets, non-amplification of controls, and contamination of negative controls.

The primary analysis first evaluates recipient and donor genomes for “basic genotyping” (bGT). The bGT process labels the donor and/or recipient with three possible genotypes at each target (e.g. homozygous AA, heterozygous AB, and homozygous BB). This information is needed in order to accurately interpret the qGT per target. Informative targets are defined as those where the recipient is known homozygous and the donor has another genotype. Where the donor is homozygous and different from the recipient, the target is referred to as fully-informative, because the observed B allele ratio is approximately the overall donor cf-DNA level. Where the donor is heterozygous, the target is called half-informative because the contribution is to both the A and B alleles, meaning the measured contribution must be doubled. For robustness, the median of informative and quality-control-passed allele ratios is reported as the percentage of donor cf-DNA.

Each qGT process yields two major quality measures, the rCV and dQC. The regularized robust coefficient of variation (rCV) is computed using the distribution of the informative and quantifiable targets. First, the robust standard deviation (rSD) is computed as the median absolute divergence from the median minor species proportion, scaled by a normalizing factor of 1.4826. The rSD is converted to a coefficient of variation by dividing by the donor cf-DNA % after it has been regularized by adding a quarter of one percent, to avoid instability around a zero divisor. The rCV measures the spread of assayed targets around their median and serves as a metric of precision or sample quality. Useful samples will generally have an rCV below 50%.

The dQC is a discordance quality check: the average minor allele proportion of recipient homozygous and non-informative targets is evaluated in order to safeguard against sample mixups and contamination. These should theoretically read nearly zero percent, subject to non-specificity allelic noise. If a sample-swap had occurred during collection or processing, the wrong recipient genotypes are used, and the dQC immediately flags up to 50 or 100% readings at presumed non-informative targets. The dQC also captures sample contamination and possibly genomic instability. Useful samples will generally have a dQC below 0.5%.

A secondary method to compute donor cf-DNA is applicable when the donor genotype is not available. Using just the recipient's genotypes and qGT results, donor options are evaluated in a Monte Carlo simulation. Preliminary random selections illustrate what overall results a given qGT sample could represent. Statistical analyses of the simulation findings provide support for probable donor genotypes. Secondary Monte Carlo simulations explore the possible or likely donor genotype space and yield a range of probable qGT outcomes. Each of 50,000 simulations reports a median Dcf-DNA, rCV and dQC triplet, and constitutes a three dimensional point cloud. The point cloud is sliced for the lower-third of dQC and rCV and the remaining “quadrant” represents the simulations corresponding to a realistic and clean sample. The central 95% of the resulting donor cf-DNA calls becomes the outcome for the qGT without the donor genotype.

### Donor Fraction

Donor fractions (or percent donor cf-DNA) were calculated and compared against events such as cellular rejection, antibody mediated rejection, graft vasculopathy, and clinically significant events of death, cardiac arrest, cardiac retransplantation, and the initiation of mechanical circulatory support. If a subject was diagnosed with cancer or post-transplant lymphoproliferative disease, or became pregnant, the first dates of diagnosis were recorded, if applicable.

Genotyping of samples from subjects passed inclusion/exclusion criteria and were used for subsequent analysis. Genotyping of each donor recipient pair resulted in informative loci per sample.

### Statistics

The median test for independent medians was performed to test whether rejection type (CR0, CR1, CR2) has equal medians by method type (with donor genotype or with simulations without donor genotype). When combining CR0 and CR1 and comparing the median of these methods to CR2, the p-values were greater than 0.05. It was, therefore, concluded that the medians are equal across rejection types. However, when comparing medians across the three rejection types (CR0 vs. CR1 vs. CR2), the p-values were less than 0.05, and it was concluded that the medians determined when the donor genotype was known and when the donor genotype was unknown are not equal with respect to rejection type.

Receiver-operating characteristic (ROC) curves were constructed to assess the sensitivity and specificity of the two analytical methods and to compare their ability to diagnose CR0 vs. CR1 vs. CR2. The optimal cutoff point or decision threshold is the point that gives maximum correct classification and the method by Liu et al. (Stat Med. 31(23):2676-86 (2012)) was used. This method maximizes the product of the sensitivity and specificity. The negative and positive predictive values of the tests were also computed. For example, a positive predictive value (PPV) of 13.4% represents that, among those who had a positive screening test, the probability of disease was 13.4%. Likewise, a negative predictive value (NPP) of 100% shows that, among those who had a negative screening test, the probability of being disease free was 100%.

### Example System Implementation

According to one embodiment of any of the systems, the system executes software to determine a donor fraction (%) where the donor genotype is unknown. In one example, the execution includes any one or more or any combination of the following operations:

1. A Monte Carlo simulation is executed across donor genotypes to determine the possible donor fraction (in other embodiments, other models or approximations may be used);

2. Two phase approach, wherein an initial short simulation of samples (e.g., of a threshold number of samples (e.g., 1000, 2000, 3000, 4000, 5000, 5999, among other options) is used to inform a secondary simulation of a larger number of samples (e.g., 10000, 15000, 20000, 25000, 29999, etc.)—in the simulation a median donor fraction, rCV and a dQC triplet can be calculated;

3. In the initial simulation the evident donor genotypes can be determined by performing a generalized linear modeling of target selections' influence on rCV and dQC separately. Further analysis of the entropy and frequency of target selections among high-background samples are added to a donor genotype likelihood offset term;

4. In the initial simulation, donor genotypes can be chosen uniformly (e.g., set as 22.7% RR, 45.5% RV, 22.7% VV, 10% NA) (e.g. heterozygous (RV), homozygous variant (VV) and homozygous reference (RR);

5. The secondary simulation chooses donor genotypes as 25% RR, 50% RV, and 75% VV, with a uniform random variable offset by the above evidence vector, less two targets for unbiasing;

6. A three-dimensional point cloud is created and a portion censored. Simulations with extreme values of median donor fraction and rCV as defined by an exponential function 0.001/3+(exp(3*x)−1)/2750 which are marked for censoring. In some embodiments, if more than 95% of the simulations are to be censored, the algorithm can be configured to recover those above their midpoint of median donor fractions;

7. Of the remaining simulations, the lower background noise simulations are identified as those below the first quartile of dQC. According to any one of the system or method embodiments, simulations above the lower quartile of dQC are discarded;

8. Of the remaining simulations, the internally consistent simulations are identified as those below the first third of rCV. According to any one of the system or method embodiments, simulations above the lower third of rCV can be discarded. In other examples, different cut offs can be implemented for rCV;

9. In any one of the system or method embodiments, a calibration can be included in the donor analysis execution, for example, the donor fractions can be scaled by a linear formula (e.g., y<−(1.166002)x+0.0001230337); and

10. In any one of the system or method embodiments, the algorithm is configured to capture the 48th percentile of median donor fractions are return that information.

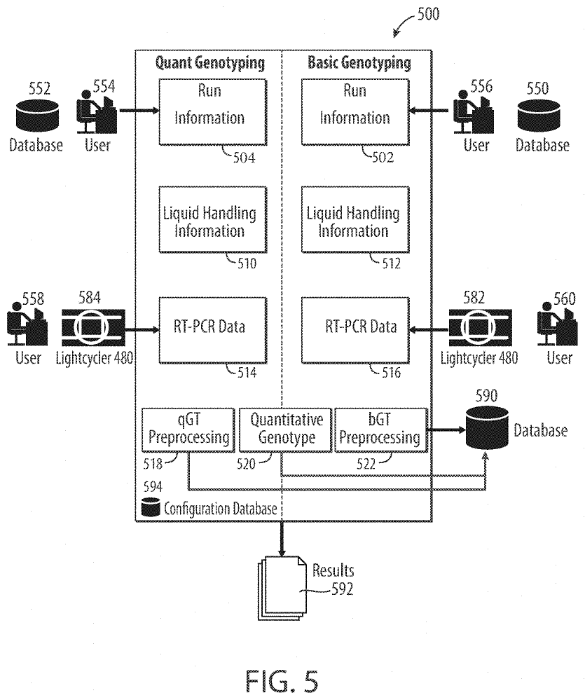

FIG. 5 is a block diagram of platform 500 including system elements and functions for analyzing a sample, according to one embodiment. In various embodiments, platform 500 can receive or generate the data to be analyzed. For example, the system can capture data from external database (e.g., 550, 552) and analyze the captured data. In other examples, users (e.g., 554, 556) can manage or trigger the communication of the data to the platform 500. In further examples, users (658, 560) can operate assay devices and/or amplification devices (e.g., 582, 584 and the results provided directly to the platform 500.

According to various embodiments of any one of the systems or methods, the analysis performed can be described by three phases: bGT preprocessing, gGT preprocessing, and quantitative genotype processing and the results output at 592 and/or stored (e.g., in database 590).

In some embodiments, run and sample information (e.g., basic genotyping run information 502 and/or quantitative genotyping run information 504) is captured through operation of a graphical user interface. In some examples, basic genotyping preprocessing operates with information which can include specification of operator name, sample identifier, and sample location; quantitative genotype preprocessing can operate with information which can include run name, operator name, sample identifier, and sample location; and outcome call processing operates with information which can include bGT preprocessing data files, file designation (recipient or donor), qGT preprocessing data files, run name and sample name. The configuration database 594 can include information specifying data format, control information, and data on other functions, including administrative functions.

Shown in FIG. 5, data from lightcycler 480 (e.g., 582 and 584) is processed as part of sample analysis. In one example, the platform 500 captures data from ROCHE Lighcycler 480 via XML files or other suitable data format. The data can be communicated with user management (e.g., triggered by users 558 or 560).

Shown in FIG. 5 at 518 are three workflows which operate on run information obtained (e.g., 506 and 508), liquid handling information (e.g., 510 and 512), and RT-PCR data (e.g., 514 and 516 (which can include, for example, real time PCR data). The three workflows include: bGT preprocessing 522 which reads data obtained on a genomic DNA sample (for example, in conjunction with a plate layout configuration information) to generate a data file (e.g., binary data file) consisting of basic genotyping results and quality control documents—these files can be archived on separate data repositories or systems; qGT preprocessing 518 which reads data on a cell-free DNA sample (for example, in conjunction with a plate layout configuration) to generate a data file (e.g., binary data file) consisting of quantitative genotyping results and quality control documents—these files can be archived on separate data repositories or systems; and quantitative genotype processing 520 where a pair of basic genotyping and quantitative genotyping data files (e.g., from 518 and 520) are analyzed to generate the outcome measures and overall quality control documents—these files can be archived on separate data source or systems including, for example, database 590. In various embodiments, the results 592 can be displayed by the platform or communicated to other systems for display.

What is claimed is:

1. A method, comprising:
analyzing amounts of alleles at multiple respective targets in a sample, and identify quantifiable and/or informative targets, within the sample;
performing simulations with possible genotypes for a non-subject; and
determining amounts of alleles of each target attributed to the non-subject and, optionally, the subject, based on probable non-subject genotype(s) determined from the simulation, and, optionally, determining a percent or ratio of non-subject to subject amounts in the sample.

2. The method of claim 1, wherein the method further comprises determining the subject genotype.

3. The method of any one of claim 1 or 2, wherein the method further comprises performing amplifications to determine the amounts of alleles.

4. The method of 3, wherein the amplifications are performed for at least 30, 40, 50, 60, 70, 80, 90, or more targets.

5. The method of any one of the preceding claims, further comprising calculating quality measures on determined percents or ratios in the sample.

6. The method of any one of the preceding claims, wherein the method comprises simulating a likely non-subject genotype space.

7. The method of any one of the preceding claims, wherein simulations (e.g., Monte Carlo) are performed to determine a range of probable genotypes for the non-subject.

8. The method of any one of the preceding claims, wherein the method further comprises adjusting measured contributions for respective targets based on respective probable genotypes (e.g., doubling measured contribution value responsive to determining the non-subject probable genotype is heterozygous).

9. The method of any one of the preceding claims, wherein the method further comprises calculating an average, such as median, percent or ratio.

10. The method of any one of the preceding claims, wherein the method further comprises determining each standard curve and/or sample amplification value meets a confidence threshold.

11. The method of any one of the preceding claims, wherein the method further comprises determining confidence values based on analysis of at least one of a historic amplification shape, specificity of the allele-specific PCR assay (e.g., with respect to a second allele), signal to noise ratio for a sample, slope and r-square value for standard curve sets, non-amplification values obtained on inserted controls, or contamination values obtained on the sample from negative controls.

12. The method of any one of the preceding claims, wherein the method further comprises fitting data obtained from the sample to a historic amplification shape.

13. The method of any one of the preceding claims, wherein the method further comprises determining the slope and r-square value for the standard curve sets does not exceed a threshold value.

14. The method of any one of the preceding claims, wherein the method further comprises establishing a label for the non-subject or subject at each target identified as quantifiable and/or informative in the sample.

15. The method of any one of the preceding claims, wherein the method further comprises determining informative targets within the sample responsive to classifying a respective target according to genotype.

16. The method of any one of the preceding claims, wherein the method further comprises classifying the respective target as informative responsive to determining the subject and non-subject have different genotypes (e.g., the subject is homozygous for one allele and the non-subject is not homozygous or homozygous for the other allele).

17. The method of any one of the preceding claims, wherein the method further comprises adjusting measured contributions for a respective target responsive to determining the non-subject is heterozygous (e.g., doubling measured contribution value responsive to determining the non-subject is heterozygous).

18. The method of any one of the preceding claims, wherein the method further comprises calculating a median of informative (e.g., identified by the genotyping component) and quality-control-passed (e.g., identified by the quality control component) allele ratios and stores the median values as the ratio or percentage.

19. The method of any one of the preceding claims, wherein the method further comprises calculating a regularized robust coefficient of variation (“rCV”) based on a distribution of the informative and quantifiable targets and associated percents or ratios.

20. The method of any one of the preceding claims, wherein the method further comprises calculating a robust standard deviation (“rSD”) based on a median absolute divergence from a median minor species proportion.

21. The method of any one of the preceding claims, wherein the method further comprises converting the rSD into rCV by division with, for example, the non-subject cf-DNA percentage.

22. The method of any one of the preceding claims, wherein the method further comprises adjusting rSD to avoid division by zero (e.g., by adding a quarter of one percent to the divisor).

23. The method of any one of the preceding claims, wherein the method further comprises identifying a sample suitable for quantification based on a threshold rCV value determined on a distribution of the informative and quantifiable targets and associated percents or ratios.

24. The method of any one of the preceding claims, wherein the method further comprises evaluating an average minor allele proportion of subject homozygous and non-informative targets against a contamination threshold.

25. The method of any one of the preceding claims, wherein the method further comprises calculating a discordance quality check (“dQC”) value based on the average minor allele proportion of the subject homozygous and the non-informative targets and evaluate the dQC value against the threshold.

26. The method of any one of the preceding claims, wherein the method further comprises identifying samples suitable for quantification based on identifying a dQC value below 0.5%

27. The method of any one of the preceding claims, wherein the non-subject is a donor.

28. The method of any one of the preceding claims, wherein the sample is from a transplant subject.

29. The method of claim 28, wherein the transplant subject is a heart transplant subject.

30. The method of claim 28 or 29, wherein the sample is from a pediatric subject.

31. The method of any one of the preceding claims, wherein the method further comprises selecting an aggregate and/or the 95% confidence interval of the probable simulations.

32. The method of any one of the preceding claims, wherein the method further comprises selecting simulations with below median dQC and rCV and/or determining the 95% confidence interval.

33. A system for analyzing a sample from a subject, the system comprising:
at least one processor operatively connected to a memory;
a first component (e.g., a quality control component), executed by the at least one processor, configured to analyze (e.g., quantitative genotyping (“qGT”)) amounts of alleles at multiple respective targets in a sample, and identify quantifiable and/or informative targets, within the sample;
a second component (e.g., a modelling component) configured to simulate possible genotype information for a non-subject; and
a third component (e.g., a genotyping component), executed by the at least one processor, configured to determine amounts of alleles of each target attributed to the non-subject and, optionally the subject, based on probable non-subject genotype(s) determined from the simulation, and, optionally, determining a percent or ratio of non-subject to subject amounts in the sample.

34. The system of claim 33, further comprising a fourth component (e.g., an analytic component), executed by the at least one processor, configured to calculate quality measures on determined percents or ratios in the sample.

35. The system of any one of claim 33 or 34, wherein the third component is configured to simulate a likely non-subject genotype space.

36. The system of any one of claims 33-35, wherein the third component is configured to execute a simulation (e.g., Monte Carlo) to determine a range of probable genotypes for the non-subject.

37. The system of any one of claims 33-36, wherein the third component is configured to adjust measured contributions for respective targets based on respective probable genotypes (e.g., doubling measured contribution value responsive to determining the non-subject probable genotype is heterozygous).

38. The system of any one of claims 33-37, wherein the at least one processor is configured to calculate an average, such as median, percent or ratio.

39. The system of any one of claims 33-38, wherein the first component is configured to determine each standard curve and/or sample amplification value meets a confidence threshold.

40. The system of any one of claims 33-39, wherein the first component is configured to determine confidence values based on analysis of at least one of a historic amplification shape, specificity of the allele-specific PCR assay (e.g., with respect to a second allele), signal to noise ratio for a sample, slope and r-square value for standard curve sets, non-amplification values obtained on inserted controls, or contamination values obtained on the sample from negative controls.

41. The system of claim 40, wherein the first component is configured to fit data obtained from the sample to a historic amplification shape.

42. The system of claim 40, wherein the first component is configured to determine the slope and r-square value for the standard curve sets does not exceed a threshold value.

43. The system of any one of claims 33-42, wherein the first or third component is configured to establish a label for the non-subject or subject at each target identified as quantifiable and/or informative in the sample.

44. The system of claim 43, wherein the first or third component is configured to determine informative targets within the sample responsive to classifying a respective target according to genotype.

45. The system of claim 43 or 44, wherein the third component is configured to classify the respective target as informative responsive to determining the subject and non-subject have different genotypes (e.g., the subject is homozygous for one allele and the non-subject is not homozygous or homozygous for the other allele).

46. The system of any one of claims 33-45, wherein the third component is configured to adjust measured contributions for a respective target responsive to determining the non-subject is heterozygous (e.g., doubling measured contribution value responsive to determining the non-subject is heterozygous).

47. The system of any one of claims 33-46, wherein the third component calculates a median of informative (e.g., identified by the genotyping component) and quality-control-passed (e.g., identified by the quality control component) allele ratios and stores the median values as the ratio or percentage.

48. The system of any one of claims 33-47, wherein any one of the components (e.g., the analytic component) is configured to calculate a regularized robust coefficient of variation (“rCV”) based on a distribution of the informative and quantifiable targets and associated percents or ratios.

49. The system of any one of claims 33-48, wherein any one of the components (e.g., the analytic component) is configured to calculate a robust standard deviation (“rSD”) based on a median absolute divergence from a median minor species proportion.

50. The system of claim 49, wherein any one of the components (e.g., the analytic component) is configured to convert the rSD into rCV by division with, for example, the non-subject cf-DNA percentage or ratio.

51. The system of claim 49 or 50, wherein the component is configured to adjust rSD to avoid division by zero (e.g. by adding a quarter of one percent).

52. The system of any one of claims 33-51, wherein the system is configured to identify a sample suitable for quantification based on a threshold rCV value determined on a distribution of the informative and quantifiable targets and associated percents or ratios.

53. The system of any one of claims 33-52, wherein the system is configured to evaluate an average minor allele proportion of subject homozygous and non-informative targets against a contamination threshold.

54. The system of claim 53, wherein the system is configured to calculate a discordance quality check (“dQC”) value based on the average minor allele proportion of the subject homozygous and the non-informative targets and evaluate the dQC value against the threshold.

55. The system of claim 53 or 54, wherein the system is configured to identify samples suitable for quantification based on identifying a dQC value below 0.5%.

56. The system of any one of claims 33-55, wherein the non-subject is a donor.

57. The system of any one of claims 33-55, wherein the sample is from a transplant subject.

58. The system of claim 57, wherein the transplant subject is a heart transplant subject.

59. The system of claim 57 or 58, wherein the sample is from a pediatric subject.

60. The system of any one of claims 33-59, wherein the system is further configured to select an aggregate and/or the 95% confidence interval of the probable simulations.

61. The system of any one of claims 33-60, wherein the system is further configured to select simulations with below median dQC and rCV and/or determining the 95% confidence interval.

62. A report comprising any one or more values that result from any one of the preceding methods or systems.

63. A method of treating a subject, comprising:
evaluating a subject based on any one or more values that result from any one of the preceding methods or systems,
and treating, recommending a treatment, changing a treatment, further monitoring or recommending further monitoring of the subject.

64. Any one of the methods as provided herein.

65. Any one of the systems as provided herein.
